(19) United States
(12) Patent Application Publication
(10) Pub. No.: US 2009/0214612 A1
(43) Pub. Date: Aug. 27, 2009

(54) EXTRACELLULAR TNF INHIBITORS FOR TREATING CNS DISORDERS

(75) Inventor: Lisa L. Shafer, Stillwater, MN (US)

Correspondence Address: K&L Gates LLP, 1900 MAIN STREET, SUITE 600, IRVINE, CA, 92614-7319, US

(73) Assignee: Medtronic Inc., Minneapolis, MN (US)

(21) Appl. No.: 12/463,065

(22) Filed: May 8, 2009

Related U.S. Application Data
(63) Continuation of application No. 10/972,177, filed on Oct. 22, 2004.
(60) Provisional application No. 60/514,137, filed on Oct. 24, 2003.

(51) Int. Cl. A61K 9/00; A61K 38/16; A61K 39/395; A61P 25/00

(52) U.S. Cl. 424/422; 514/12; 424/158.1; 424/145.1; 424/134.1; 424/133.1; 424/142.1

(57) ABSTRACT

Methods and devices to attenuate tumor necrosis factor (TNF) and other pro-inflammatory mediators in the CNS to treat neurological, neurodegenerative, neuropsychiatric disorders, and brain injury are described. More particularly, TNF blocking agents that target TNF-receptor interactions and the effects of downstream secreted cytokines associated with an inflammatory cascade are described. Such TNF blocking agents are administered directly to the brain by, for example, intraparenchymal administration, intracerebroventricular administration, or administration into a cerebral artery. Devices described include therapy delivery devices comprising a reservoir capable of housing a TNF blocking agent and a catheter operably coupled to the device and adapted to deliver the TNF blocking agent to a target site within a subject.

### RELATED APPLICATIONS

This application is a continuation of U.S. patent application Ser. No. 10/972,177 filed Oct. 22, 2004 which claims the benefit of priority to Provisional Application Ser. No. 60/514,137, filed Oct. 24, 2003, both of which applications are incorporated herein by reference in their entirety.

### FIELD

This invention relates to medical devices and methods for attenuating pro-inflammatory mediators, particularly for treatment of neurological, neurodegenerative, neuropsychiatric disorders, and brain injury.

### BACKGROUND

Neurodegeneration that is characteristic of neurodegenerative disease and traumatic brain injury may progress even when the initial cause of neuronal degeneration or insult has disappeared. It is believed that toxic substances released by the neurons or glial cells may be involved in the propagation and perpetuation of neuronal degeneration. Neuronal degeneration and other disease pathology in the brain has been attributed to the toxic properties of proinflammatory cytokines, such as tumor necrosis factor alpha or beta (TNF), interleukin (IL)-1 beta, and interferon (IFN)-gamma. Therapies aimed at inhibiting proinflammatory cytokines, particularly TNFα, may attenuate the pathology associated with chronic pain, neurodegenerative diseases, traumatic brain injury and abnormal glial physiology. Furthermore, inhibiting the constitutive levels of pro-inflammatory cytokines may provide a prophylactic therapy for individuals at risk for, or at early stages of, a certain disease or condition of the brain.

Several TNF blocking agents have been developed for systemic administration and are approved for treating various diseases of the periphery such as rheumatoid arthritis and Crohn's disease. Currently available blocking agents act on soluble, extracellular TNF or TNF receptors. These agents are administered in the periphery and are not capable of penetrating the blood-brain-barrier. While these agents are effective for the above-mentioned indications, this class of TNF blocking agents is associated with the risk of serious side-effects, such as opportunistic infections, immuno-supression and demyelinating diseases. Moreover, recent reports have led to the counter-indication of systemic, chronic use of some of the commercially available TNF blocking agents in individuals with a history of central nervous system disorders.

Despite this counter-indication, the use of such TNF blocking agents to treat neurological and neuropsychiatric disorders has recently been suggested. US 2003/0049256A1 and WO 04/032718 (Tobinick) discuss the administration of cytokine antagonists via intranasal and perispinal routes of administration as a way of treating neurological or neuropsychiatric disorders or diseases. The Tobinick patents do not disclose targeted administration of such agents intraventricularly or to the intraparenchymal brain tissue.

Many TNF blocking agents that blocking extracellular TNF and its extracellular or cell surface receptors form complexes composed of soluble TNF and its blocking agent. In the periphery, these complexes are broken down and eliminated via phagocytic clearance. This mechanism of action is efficacious and therapeutic in several peripheral diseases. However, the brain does not have these same clearance mechanisms. Therefore, it is possible that there is a greater potential for the toxic TNF molecule to be stabilized by the blocking agents, leading to greater toxic effects in the brain tissue.

Furthermore, in the periphery, some currently available blocking agents ultimately engage the TNF receptor and initiate apoptosis, or programmed cell death, in the TNF producing cell. This is a desired effect of a TNF blocking therapy in the periphery because death of activated cells is beneficial and because these cells are capable of replenishing themselves. However, when these same agents are applied to cells of the central nervous system (CNS) and their mechanism of action results in apoptosis of neurons, a deleterious effect can occur. Because neurons are substantially incapable of regenerating themselves, apoptosis of neurons is detrimental to the brain.

Moreover, since several different brain cell types produce TNF and express TNF receptors, the indiscriminant blocking of TNF receptors on a cell surface may result in non-target cell tissue binding. This non-specific effect may have serious consequences in the brain. Compared to the periphery, brain tissue is less “immunocompetent” and as a result, this non-specific effect cannot be compensated for and may result in exacerbated conditions.

TNFα is a non-glycosylated polypeptide that exists as either a transmembrane or soluble protein. TNFα increases production of pro-inflammatory molecules and several adhesion molecules resulting in the initiation of an inflammatory cascade, which can result in the release of additional inflammatory cytokines. Frequently, the TNF-initiated cascade has deleterious effects at the cellular, tissue and organ level.

TNF and TNF receptors are expressed in the brain by astrocytes, neurons, monocytes, microglia and blood vessels. Biologic or small molecule drug therapeutic agents targeting the extracellular TNF cascade, including TNF-receptor binding and the action of released cytokines in these cell populations may have a therapeutic or prophylactic effect in diseases and conditions of the central nervous system. However, the administration of such extracellular TNF blocking agents has not been previously described.

### BRIEF SUMMARY

This disclosure describes the delivery of extracellular TNF and other cytokine blocking agents (collectively referred to herein as “extracellular TNF blocking agents”) directly to the brain to treat a central nervous system (CNS) disorder in a subject in need thereof. Despite the potential adverse effects of such agents to the CNS, targeted delivery directly to the brain should minimize the adverse effects and allow for improved treatment of CNS disorders associated with inflammation or inflammatory cytokines.

An embodiment of the invention provides a method for treating a CNS disorder in a subject in need thereof. The method comprises administering an extracellular TNF blocking agent directly to the subject's brain. The extracellular TNF blocking agent may be administered intracerebroventricularly, into a cerebral artery, or intraparenchymally. The agent may be administered via a catheter. The catheter may have a delivery portion, which is positioned within a targeted region of the brain. The catheter may be operably coupled to a therapy delivery device, such as a pump system. The method may be used to treat a variety of CNS disorders, including neurological disorders, neurodegenerative disorders, neuropsychiatric disorders, and brain injury. Exemplary disorders that may be treated include stroke, Alzheimer's disease, epilepsy, and stroke.

Various embodiments of the invention may provide one or more advantages. For example, as discussed herein, direct, targeted administration of extracellular TNF blocking agents to the brain may minimize side effects associated with more global administration of such blocking agents. Use of a programmable pump system for such targeted delivery may allow for further reduction in side effects as the delivery dose may be readily titrated to balance efficacy against side effects. Further more, targeted delivery generally may allow for improved efficacy while minimizing potential side effects by maximizing the concentration of the extracellular TNF agent to a brain region to be treated and reducing concentrations in regions not to be treated. These and other advantages will become evident to those of skill in the art upon reading the description provided herein.

### BRIEF DESCRIPTION OF THE DRAWINGS

FIG. 1 is a schematic diagram of TNF signal transduction.

FIG. 2 is a diagrammatic illustration of a drug delivery system according to an embodiment of the present invention.

FIG. 3 is a diagrammatic illustration of a catheter implanted in a patient and a drug delivery system according to an embodiment of the present invention.

FIG. 4 is a diagrammatic illustration of a drug delivery system and catheter implanted in a patient according to an embodiment of the present invention.

FIG. 5 is a diagrammatic illustration of a drug delivery system comprising a sensor according to an embodiment of the present invention.

The figures are not necessarily to scale.

### DETAILED DESCRIPTION OF THE INVENTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like mean alleviating, slowing the progression, preventing, attenuating, or curing the treated disease.

As used herein, “disease”, “disorder”, “condition” and the like, as they relate to a subject's health, are used interchangeably and have meanings ascribed to each and all of such terms.

As used herein, “subject” means a mammal undergoing treatment. Mammals include mice, rats, cats, guinea pigs, hamsters, dogs, horses, cows, monkeys, chimpanzees, and humans.

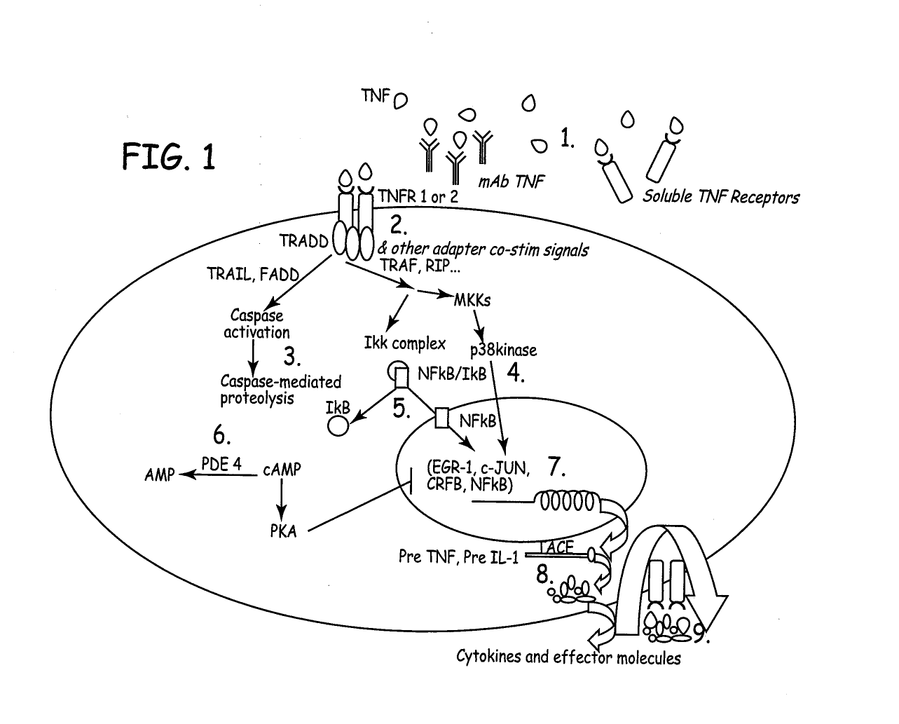

As used herein, “extracellular TNF blocking agent” means an agent that affects the action of TNF at a TNF cell surface receptor and agents that affect the action of secreted molecules associated with the TNF inflammatory cascade, such as IL-1, IL-6, and HMG-B1. Extracellular TNF blocking agents include small molecule chemical agents and biological agents, such as polynucleotides and polypeptides, which include antibodies and fragments thereof, antisense, small interfering RNA (siRNA), and ribosymes. Nonlimiting examples of extracellular TNF blocking agents include agents that act at sites 1 and 9 shown in FIG. 1.

Delivery System

An embodiment of the invention provides a system for delivering a therapeutic composition comprising an extracellular TNF blocking agent to a CNS of a subject in need thereof. The system comprises therapy delivery device and a catheter operably coupled to the therapy delivery device. The therapy delivery device may be a pump device. Non-limiting examples of pump devices include osmotic pumps, fixed-rate pumps, programmable pumps and the like. Each of the aforementioned pump systems comprise a reservoir for housing a fluid composition comprising an extracellular TNF blocking agent. The catheter comprises one or more delivery regions, through which the fluid may be delivered to one or more target regions of the subject. The pump device may be implantable or may be placed external to the subject.

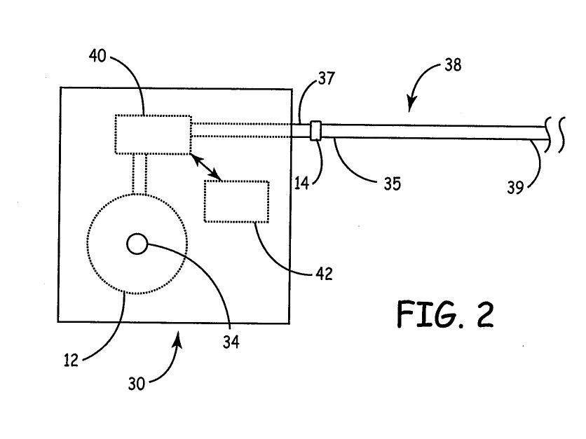

The therapy delivery device 30 shown in FIG. 2 comprises a reservoir 12 for housing a composition comprising an extracellular TNF blocking agent and a pump 40 operably coupled to the reservoir 12. The catheter 38 shown in FIG. 2 has a proximal end 35 coupled to the therapy delivery device 30 and a distal end 39 adapted to be implanted in a subject. Between the proximal end 35 and distal end 39 or at the distal end 39, the catheter 38 comprises one or more delivery regions (not shown) through which the extracellular TNF blocking agent may be delivered. The therapy delivery device 30 may have a port 34 into which a hypodermic needle can be inserted to inject a quantity of extracellular TNF blocking agent into reservoir 12. The therapy delivery device 30 may have a catheter port 37, to which the proximal end 35 of catheter 38 may be coupled. The catheter port 37 may be operably coupled to reservoir 12. A connector 14 may be used to couple the catheter 38 to the catheter port 37 of the therapy delivery device 30. The therapy delivery device 30 may be operated to discharge a predetermined dosage of the pumped fluid into a target region of a patient. The therapy delivery device 30 may contain a microprocessor 42 or similar device that can be programmed to control the amount of fluid delivery. The programming may be accomplished with an external programmer/control unit via telemetry. A controlled amount of fluid comprising a extracellular TNF blocking agent may be delivered over a specified time period. With the use of a programmable delivery device 30, different dosage regimens may be programmed for a particular patient. Additionally, different therapeutic dosages can be programmed for different combinations of fluid comprising therapeutics. Those skilled in the art will recognize that a programmed therapy delivery device 30 allows for starting conservatively with lower doses and adjusting to a more aggressive dosing scheme, if warranted, based on safety and efficacy factors.

If it is desirable to administer more than one therapeutic agent, such as one or more TNF blocking agent, the fluid composition within the reservoir 12 may contain a second, third, fourth, etc. therapeutic agent. Alternatively, the device 30 may have more than one reservoir 12 for housing additional compositions comprising a therapeutic agent. When the device 30 has more than one reservoir 12, the pump 40 may draw fluid from one or more reservoirs 12 and deliver the drawn fluid to the catheter 38. The device 30 may contain a valve operably coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the pump 40. A catheter 38 may have more than one lumen. Each lumen may be adapted to deliver a therapeutic agent from one or more reservoirs 12 of the device 30. It will also be understood that more than one device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

According to an embodiment of the invention, a composition comprising an extracellular TNF blocking agent may be delivered intracerebroventricularly or intraparenchymally directly to brain tissue of a subject. A therapy delivery device may be used to deliver the agent to the brain tissue. A catheter may be operably coupled to the therapy delivery device and a delivery region of the catheter may be placed in or near a target region of the brain.

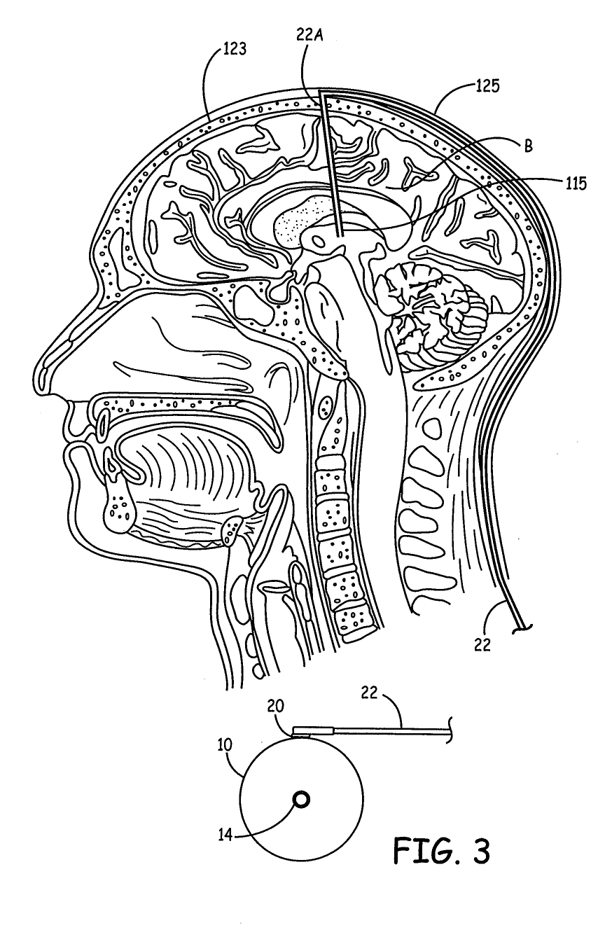

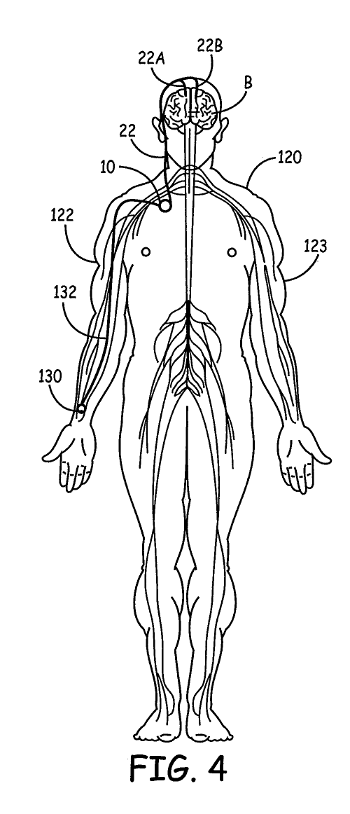

One suitable system for administering a therapeutic agent to the brain is discussed in U.S. Pat. No. 5,711,316 (Elsberry) as shown FIGS. 3 and 4 herein. Referring to FIG. 3, a system or therapy delivery device 10 may be implanted below the skin of a patient. The device 10 may have a port 14 into which a hypodermic needle can be inserted through the skin to inject a quantity of a composition comprising a therapeutic agent. The composition is delivered from device 10 through a catheter port 20 into a catheter 22. Catheter 22 is positioned to deliver the agent to specific infusion sites in a brain (B). Device 10 may take the form of the like-numbered device shown in U.S. Pat. No. 4,692,147 (Duggan), assigned to Medtronic, Inc., Minneapolis, Minn. The distal end of catheter 22 terminates in a cylindrical hollow tube 22A having a distal end 115 implanted into a target portion of the brain by conventional stereotactic surgical techniques. Additional details about end 115 may be obtained from pending U.S. application Ser. No. 08/430,960 entitled “Intraparenchymal Infusion Catheter System,” filed Apr. 28, 1995 in the name of Dennis Elsberry et at. and assigned to the same assignee as the present application. Tube 22A is surgically implanted through a hole in the skull 123 and catheter 22 is implanted between the skull and the scalp 125 as shown in FIG. 1. Catheter 22 is joined to implanted device 10 in the manner shown, and may be secured to the device 10 by, for example, screwing catheter 22 onto catheter port 20.

Referring to FIG. 4, a therapy delivery device 10 is implanted in a human body 120 in the location shown or may be implanted in any other suitable location. Body 120 includes arms 122 and 123. Catheter 22 may be divided into twin tubes 22A and 22B that are implanted into the brain bilaterally. Alternatively, tube 22B may be supplied with drugs from a separate catheter and pump.

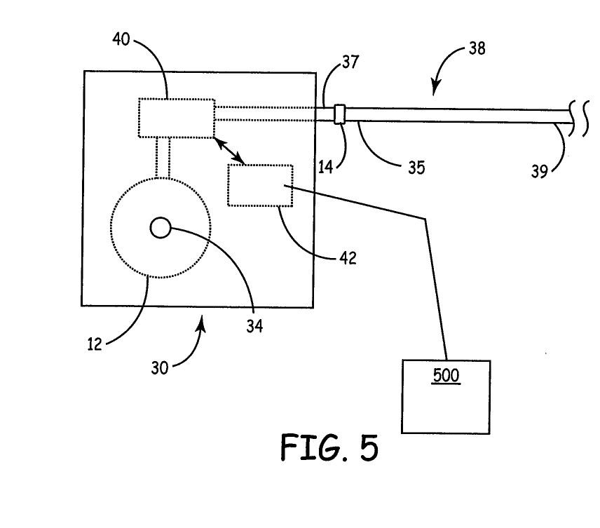

Referring to FIG. 5, therapy delivery device 30 may include a sensor 500. Sensor 500 may detect an event associated with a CNS disorder associated with an inflammatory immune response, such as a dysfunctional immune or sickness response, or treatment of the disorder, such as or whether an immune response has been attenuated or enhanced. Sensor 500 may relay information regarding the detected event, in the form of a sensor signal, to processor 42 of device 30. Sensor 500 may be operably coupled to processor 42 in any manner. For example, sensor 500 may be connected to processor via a direct electrical connection, such as through a wire or cable. Sensed information, whether processed or not, may be recoded by device 30 and stored in memory (not shown). The stored sensed memory may be relayed to an external programmer, where a physician may modify one or more parameter associated with the therapy based on the relayed information. Alternatively, based on the sensed information, processor 42 may adjust one or more parameters associated with therapy delivery. For example, processor 42 may adjust the amount and timing of the infusion of a TNF blocking agent. Any sensor 500 capable of detecting an event associated with an the disease to be treated or an inflammatory immune response may be used. Preferably, the sensor 500 is implantable. It will be understood that two or more sensors 500 may be employed.

Sensor 500 may detect a polypeptide associated with a CNS disorder or an inflammatory immune response; a physiological effect, such as a change in membrane potential; a clinical response, such as blood pressure; and the like. Any suitable sensor 500 may be used. In an embodiment, a biosensor is used to detect the presence of a polypeptide or other molecule in a patient. Any known or future developed biosensor may be used. The biosensor may have, e.g., an enzyme, an antibody, a receptor, or the like operably coupled to, e.g., a suitable physical transducer capable of converting the biological signal into an electrical signal. In some situations, receptors or enzymes that reversibly bind the molecule being detected may be preferred. In an embodiment, sensor 500 is capable of detecting an inflammatory cytokine. In an embodiment sensor 500 is capable of detecting TNF in cerebrospinal fluid. In an embodiment, sensor 500 may be a sensor as described in, e.g., U.S. Pat. No. 5,978,702, entitled TECHNIQUES OF TREATING EPILEPSY BY BRAIN STIMULATION AND DRUG INFUSION, which patent is hereby incorporated herein by reference in its entirety, or U.S. patent application Ser. No. 10/826,925, entitled COLLECTING SLEEP QUALITY INFORMATION VIA A MEDICAL DEVICE, filed Apr. 15, 2004, which patent application is hereby incorporated herein by reference in its entirety, or U.S. patent application Ser. No. 10/820,677, entitled DEVICE AND METHOD FOR ATTENUATING AN IMMUNE RESPONSE, filed Apr. 8, 2004.

In an embodiment, cerebrospinal levels of TNF are detected. A sample of CSF may be obtained and the levels of TNF in the sample may be detected by Enzyme-Linked Immunoabsorbant Assay (ELISA), microchip, conjugated fluorescence or the like. Feedback to a therapy delivery device may be provided to alter infusion parameters of the TNF blocking agent.

TNF Blocking Agents

An embodiment of the invention provides a method for treating a CNS disorder associated with a pro-inflammatory agent by administering to the subject a composition comprising an extracellular TNF blocking agent. Such agents act primarily at sites 1 and 9 of FIG. 1.

1. Soluble TNF Inhibitors

Soluble TNF inhibitors whose site of action is at site 1 of FIG. 1 may be used to treat a CNS disorder. Examples of soluble TNF inhibitors include fusion proteins (such as etanercept); monoclonal antibodies (such as infliximab and D2E7); binding proteins (such as onercept); antibody fragments (such as CDP 870); CDP571 (a humanized monoclonal anti-TNF-alpha IgG4 antibody), soluble TNF receptor Type I, pegylated soluble TNF receptor Type I (PEGs TNF-R1) and dominant-negative TNF variants, such as DN-TNF and including those described by Steed et al. (2003), “Inactivation of TNF signaling by rationally designed dominant-negative TNF variants”, Science, 301 (5641): 1895-8. As soluble TNF inhibitor may be administered directly to a subject's brain to treat a CNS disorder.

2. Inhibition of TNFα-Post Translational Effects

The initiation of TNFα signaling cascade results in the enhanced production of numerous factors that subsequently act in a paracrine and autocrine fashion to elicit further production of TNFα as well as other pro-inflammatory agents (IL-6, IL-1, HMG-B1), as shown at site 9 of FIG. 1. Extracellular TNF blocking agents that act on the signals downstream of TNF are being developed clinically for systemic inflammatory diseases. Some of these agents are designed to block other effector molecules while others block the cellular interaction needed to further induce their production (integrins, cell adhesion molecules etc). While their use outside of the brain to modulate TNFα-induced inflammatory cascade has been suggested previously, the administration of these agents to the brain with the use of targeted drug delivery systems to treat neurological, neuropsychiatric and neurodegenerative diseases has not been described.

An embodiment of the invention provides for the selection of an agent to inhibit the TNF-induced effects that are downstream of any TNF/TNFR complex effects. This agent is then delivered to the patient, to e.g. a specific brain region, using a drug delivery system to treat neurological, neuropsychiatric and neurodegenerative diseases. The agent may be selected from the following: integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL15 antibody).

Injectable Composition

The above-mentioned TNF blocking agents may be administered to a subject's CNS as injectable compositions. Injectable compositions include solutions, suspensions, dispersions, and the like. Injectable solutions or suspensions may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Solutions or suspensions comprising a therapeutic agent may be prepared in water, saline, isotonic saline, phosphate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is sterile, fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption—for example, aluminum monosterate hydrogels and gelatin. Excipients that increase solubility, such as cyclodextran, may be added.

Sterile injectable solutions may be prepared by incorporating a therapeutic agent in the required amount in the appropriate solvent with various other ingredients as enumerated above and, as required, followed by sterilization. Any means for sterilization may be used. For example, the solution may be autoclaved or filter sterilized. In the case of sterile powders for the preparation of sterile injectable solutions, the preferred methods of preparation are vacuum drying and freeze-drying techniques, which yield a powder of the active ingredient plus any additional desired ingredient present in a previously sterile-filtered solution.

Dosage

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical composition is administered, the interaction between the same is considered and the dosages calculated.

CNS Disorder

Embodiment of the invention provide methods and devices for treating a CNS disorder associated with a pro-inflammatory agent by administering to a subject a CNS disorder treating effective amount of a composition comprising an extracellular TNF blocking agent. CNS disorders associated with a pro-inflammatory agent include neurological, neurodegenerative, neuropsychiatric disorders, and brain injury. The extracellular TNF blocking agent may be administered directly to the brain of the subject by, e.g., intracerberalventricular (ICV) delivery, intraparenchymal (IPA) delivery, or delivery into a cerebral artery. Targeted delivery to the CNS avoids the potential for systemic immuno-suppression and other risk factors associated with systemic exposure to TNF blocking agents. In various embodiments, the extracellular TNF blocking agent is delivered to the CNS using a programmable pump, which allows for controlling the rate and time at which the agent is delivered and provides the ability to stop the delivery of the agent as desired.

Examples of various CNS disorders that may be treated and preferred delivery locations of therapeutic agents for treating the disorders is provided below.

1. Stroke

Blood-brain barrier breakdown and inflammation is observed in brain following stroke. Inflammatory processes are at least partly responsible for this breakdown. TNF blocking agents may be administered ICV, either chronically or transiently, following a stroke. In an embodiment, a TNF blocking agent is administered at the location of an infarct due to stroke. The location of the infarct may be identified by MRI or other known or future developed techniques. In an embodiment, the therapeutic agent is delivered to the middle cerebral artery at an infarct location or other cerebral artery distribution. Such delivery can be accomplished by placing a delivery region of a catheter in the artery and delivering the agent through the delivery region.

In addition to the ICV delivery of a TNF blocking agent at or near an infarct, a TNF blocking agent may be delivered IPA to an area surrounding the infarct to attenuate inflammation occurring in the ischemic periphery or penumbra that may lead to neurodegeneration if left untreated.

To attenuate the degeneration that occurs in a patient with hemiperesis following stroke a TNF blocking agent may be placed in the posterior limb of the internal capsule, for example.

In addition, a TNF blocking agent may be delivered to other brain regions that may be affected due to the secondary ischemic events following stroke, including but not limited to the pons, midbrain, medulla and the like.

Additional locations where a TNF blocking agent may be administered to treat stroke include locations where inflammatory events secondary to the initial stroke may occur. For example middle cerebral artery stroke can produce a characteristic, cell-type specific injury in the striatum. Transient forebrain ischemia can lead to delayed death of the CAl neurons in the hippocampus. Therefore, a TNF blocking agent may be delivered to the striatum or hippocampus following a stroke event.

2. Alzheimer's Disease

Brain microvessels from Alzheimer's disease (AD) patients have been shown to express high levels of pro-inflammatory cytokines. It is suggested that inflammatory processes in the brain vasculature may contribute to plaque formation, neuronal cell death and neurodegeneration associated with AD. Accordingly, targeted delivery of a TNF blocking agent to a patient suffering from AD is contemplated herein.

In an embodiment, the TNF blocking agent is delivered in the vicinity of an amyloid plaque, where the inflammatory response in AD is mainly located. A TNF blocking agent may be administered IPA at the site of amyloid beta peptide accumulations, amyloid beta plaques, neurofibrillary tangles or other pathological sites associated with AD. For example, the affected area may be cortical or cerebellar and the plaques may be observed by imaging techniques known in the field.

Other IPA sites include the basal forebrain cholinergic system, a region that is vulnerable to degeneration in AD, the structures of the temporal lobe region, a region that is responsible for cognitive decline in AD patients, specifically the hippocampus, entorhinal cortex, and dentate gyrus.

3. Epilepsy

Blood-brain barrier breakdown and inflammation is observed in brain following seizures. Inflammatory processes are at least partly responsible for this breakdown. In addition, TNF production is up-regulated during seizure-induced neuronal injury. In an embodiment TNF blocking agents are administered ICV, either chronically or transiently, following a seizure episode. In an embodiment, a TNF blocking agent is administered IPA to a seizure focus. In an embodiment, a TNF blocking agent is administered IPA to an area of the brain that undergoes neuronal injury, away from a specific seizure focus. For example, in patients with intractable temporal lobe epilepsy, the CA1 region of the hippocampus undergoes pathophysiological changes associated with inflammatory processes and may ultimately result in neuronal cell loss in that region. Therefore, TNF blocking agents may be administered to the hippocampus in a epileptic patient. Other sites of IPA delivery are associated with brain regions affected by mesial temporal sclerosis such as the hippocampus or amygdala where evidence of inflammatory processes are often detected. Other structures in the CNS known to play a key role in the epileptogenic network such as the thalamus and subthalamic nucleus may also be targeted.

4. Depression

A TNF blocking agent may be administered ICV to target brain regions associated with inflammation in patients with depression. One suitable ICV location is the floor of the fourth ventricle, dorsal to the abducens nuclei, that contains serotonergic neurons.

In an embodiment, a TNF blocking agent is administered IPA to brain regions associated with the hypothalamic-pituitary-adrenal (HPA)-axis, as dysfunction of the HPA-axis is common in patients with depression. Furthermore, the cellular immune status in the brain regions associated with the HPA-axis is abnormal and is believed to be partly responsible for depressive symptoms. Elevations in proinflammatory cytokines such as TNF often found in depressed patients likely affect the normal functioning of the HPA axis. Examples of brain regions associated with the HPA-axis include, but are not limited to, the hypothalamus and the anterior pituitary gland.

In an embodiment, a TNF blocking agent is delivered to a brain region associated with serotonin production and output, since pro-inflammatory cytokines such as TNF may lower the circulating levels of serotonin-the mood stabilizing neurotransmitter. A TNF blocking agent delivered in a controlled fashion to the site of serotonin production may serve to regulate the production of serotonin thereby modulating the levels of serotonin production in patients with depression. The main site of serotonin production in the brain is the dorsal raphe nucleus. Other clusters or groups of cells that produce serotonin located along the midline of the brainstem may be targeted with IPA delivery of a TNF blocking agent. Main serotonergic nuclei may be targeted including the ventral surface of the pyramidal tract, the nucleus raphe obscurans, the raphe at the level of the hypoglossal nucleus, at the level of the facial nerve nucleus surrounding the pyramidal tract, the pontine raphe nucleus, above and between the longitudinal fasiculi at the central substantia grisea, the medial raphe nucleus, or the medial lemniscus nucleus.

All patents and publications referred to herein are hereby incorporated by reference in their entirety.

The teachings of the following patents and publications may be readily modified in light of the disclosure presented herein to produce the various devices described herein and to practice the various methods described herein:
Hirsch et al. (2003), “The role of glial reaction and inflammation in Parkinson's disease”, Ann. N.Y. Acad. Sci.; 991: 214-28.
Ito, H. (2003), “Anti-interleukin-6 therapy for Crohn's disease”, Curr. Pharm. Des.; 9(4): 295-305.
WO 03/070897 RNA Interference Mediated Inhibition of TNF and TNF Receptor Superfamily Gene Expression Using Short Interfering Nucleic Acid (siNA)
U.S. Pat. No. 2003/0185826 Cytokine antagonists for the treatment of localized disorders
U.S. Pat. No. 6,596,747, Compounds derived from an amine nucleus and pharmaceutical compositions comprising same
U.S. Pat. No. 6,180,355, Method for diagnosing and treating chronic pelvic pain syndrome
WO2003/072135, INHIBITION OF INFLAMMATORY CYTOKINE PRODUCTION BY STIMULATION OF BRAIN MUSCARINIC RECEPTORS
WO98/20868, GUANYLHYDRAZONES USEFUL FOR TREATING DISEASES ASSOCIATED WITH T CELL ACTIVATION
WO2002100330, METHODS OF ADMINISTERING ANTI-TNFα ANTIBODIES

What is claimed is:

1. A method for treating a CNS disorder associated with a proinflammatory agent in a subject in need thereof, the method comprising: administering an extracellular TNF blocking agent to the subject's brain in an amount effective to treat the CNS disorder when administered to the brain.

2. The method of claim 1, wherein administering the agent directly to the subject's brain comprises administering the agent by an administration route selected from the group consisting of intraparenchymally, intracerebroventricularly, and into a cerebral artery.

3. The method of claim 1, wherein the CNS disorder is a neurological disorder, a neurodegenerative disorder, a neuropsychiatric disorder or brain injury.

4. The method of claim 1, wherein the CNS disorder is stroke.

5. The method of claim 4, wherein administering the agent to a cerebral artery comprises administering the agent to the middle cerebral artery such as at or near an infarct.

6. The method of claim 4, wherein administering an extracellular TNF blocking agent comprises administering the agent intraparenchymally to a location selected from the group consisting of, to the hippocampus, to the CA1 region of the hippocampus, to the striatum, to the posterior limb of the internal capsule, and at or near an infarct.

7. The method of claim 1, wherein the CNS disorder is Alzheimer's disease.

8. The method of claim 7, wherein administering the agent intraparenchymally comprises administering the agent to a location selected from the group consisting of, at or near an amyloid beta plaque, to the basal forebrain cholinergic region, to the temporal lobe region, to the hippocampus, to the entorhinal cortex, and to the dentate gyrus.

9. The method of claim 1, wherein the CNS disorder is epilepsy.

10. The method of claim 9, wherein the administering the agent intraparenchymally comprises administering the agent to a location selected from the group consisting of at or near an epileptic focus, to the hippocampus, to the CA1 region of the hippocampus

11. The method of claim 1, wherein the CNS disorder is depression.

12. The method of claim 11, wherein the administering the agent intracerebroventricularly comprises administering the agent to the floor of the fourth ventricle, dorsal to the abducens nuclei.

13. The method of claim 11, wherein the administering the agent intraparenchymally comprises administering the agent to a brain region associated with the hypothalamic-pituitary-adrenal (HPA)-axis, to a brain region associated with serotonin production or output.

14. The method of claim 11, wherein administering the agent to a brain region associated with the HPA-axis comprises administering the agent to the hypothalamus or to the anterior pituitary gland.

15. The method of claim 11, wherein administering the agent to a brain region associated with serotonin production or output comprises administering the agent to a location selected from the group consisting of the dorsal raphe nucleus, to the midline of the brainstem, the ventral surface of the pyramidal tract, the nucleus raphe obscurans, the raphe at the level of the hypoglossal nucleus, at the level of the facial nerve nucleus surrounding the pyramidal tract, the pontine raphe nucleus, above and between the longitudinal fasiculi at the central substantia grisea, the medial raphe nucleus, and the medial lemniscus nucleus.

16. The method of claim 1, wherein administering the extracellular TNF blocking agent comprises administering an agent selected from the group consisting of TNF fusion protein, an antibody directed to TNF, a monoclonal antibody directed to TNF, a TNF binding protein, a soluble TNF receptor, a soluble pegylated TNF receptor, an antibody fragment directed to TNF, a dominant-negative TNF variant, an integrin antagonists, alpha-4 beta-7 integrin antagonists, a cell adhesion inhibitor, interferon gamma antagonists, a CTLA4-Ig agonists/antagonists, a CD40 ligand antagonists, a anti-IL-6 antibody, an anti-HMGB-1 antibody, an anti-IL2R antibody, an anti-IL-8 antibody, an anti-IL-10 antibody, etanercept, infliximab, D2E7, onercept, CDP 870, CDP 571, PEGs TNF-R1, DN-TNF, BMS-188667, tocilizumab (Chugai), daclizumab, basilicimab, ABX (anti IL-8 antibody), and HuMax IL-15 (anti-IL15 antibody).

17. The method of claim 1, wherein administering an extracellular TNF blocking agent to the subject's brain comprises placing a delivery region of a catheter in a target location in the subject's brain and delivering the agent through the delivery region to the target location.

18. The method of claim 17, further comprising delivering the agent from a pump through the catheter.

19. The method of claim 18, further comprising implanting the pump in the subject.

20. The method of claim 19, further comprising sensing a mediator of an inflammatory response and modifying the rate of delivery of the agent based on a signal obtained from the sensing.
